(19) United States
(12) Patent Application Publication
(10) Pub. No.: US 2015/0125537 A1
(43) Pub. Date: May 7, 2015

(54) BONE FILLER MATERIAL AND METHODS OF USE

(71) Applicants: Agency for Science, Technology and Research, Singapore (SG); National University of Singapore, Singapore (SG)

(72) Inventors: Cho Hong James Goh, Singapore (SG); Pujiang Shi, Singapore (SG); Hee Kit Wong, Singapore (SG); Yong Zhang, Singapore (SG); Sunny Akogwu Abbah, Singapore (SG); Jun Li, Singapore (SG)

(21) Appl. No.: 14/535,568

(22) Filed: Nov. 7, 2014

(30) Foreign Application Priority Data
Nov. 7, 2013 (SG) 201308278-9

(51) Int. Cl. A61L 27/54; A61L 27/50; A61L 27/34; A61L 27/56

(52) U.S. Cl. CPC A61L 27/54; A61L 27/56; A61L 27/50; A61L 27/34; A61L 2430/02; A61L 2300/604; A61L 2300/606
USPC 424/491; 424/490; 514/16.7; 424/682; 514/8.8; 424/602

(57) ABSTRACT

There is provided a bone filler material comprising a plurality of particles, each particle comprising a biodegradable outer shell and an inner core. There is also provided an implant prepared from the same, methods of using the same and preparing the same.

### CROSS-REFERENCE TO RELATED APPLICATIONS

This application claims the benefit of priority of Singapore patent application no. 201308278-9, filed Nov. 7, 2013, the contents of it being hereby incorporated by reference in its entirety for all purposes.

### TECHNICAL FIELD

The present invention generally relates to scaffolds for hard tissue. More particularly, the present invention relates to bone scaffolds and filler materials.

### BACKGROUND

In spinal fusion and regeneration of bone defects, control of deep seated blood loss is challenging. Generally, the options available to surgeons today in arresting bleeding from bony surfaces at a site of fusion or bone reconstruction can be categorized into four main groups:

1. Physical pressure with sterile gauze coupled with suction. This is always used when possible (especially in open surgery) and may be effective. However, it might not always be sufficient depending on the location and surface area of the bleeding bone.

2. Tamponade effect of bulk materials, such as tightly fitting block implants (e.g. Bagby and Kuslich (BAK) cages, autograft bone), and/or void fillers, such as calcium phosphate derivatives. These are often sufficient but may not always be suitable in applications such as multi-segmental spinal fusion which are necessary in scoliosis patients. Moreover, the advent of more advanced strategies in bone healing involving growth factors and optimized scaffolds/carrier systems demand complementary advances in delivery and localization of these biological factors.

3. Water soluble hemostatic agents such as alkylene products, microfibrillar collagen and gelatin/thrombin products. Some of these agents such as microfibrillar collagen hemostat (Colgel) and oxidized cellulose (Surgicel) have been useful in arresting bleeding in many clinical scenarios, particularly in high risk cardiac and hepatic surgeries. However, many of the current products do not possess bulk properties and often do not actively enhance bone regeneration as they are neither osteoconductive nor osteoinductive. Indeed, some of the water soluble hemostatic agents have been reported to inhibit bone formation instead. Furthermore, the handling properties of some of the water soluble products are not suitable for use in bone bleeding. For example, oxidized regenerated cellulose (e.g. Surgicel, Oxycel) requires an absolutely dry operative field. While this is achievable in soft tissue bleeding, bleeding from trabecular bone would pose a real technical challenge.

4. Bone wax, a non-water soluble hemostatic agent. Intra-operative bleeding from bony surfaces has been controlled by firm application of bone wax. Traditional bone wax is formulated from beeswax, parraffin and isopropyl palmitate or Vaseline. However, bone wax is not degradable and is known to physically inhibit bone growth and new bone formation and therefore is contra-indicated in situations where bone regeneration is desirable. As bone wax is a foreign body particle, it may detach more easily and embolise in circulation, or even form granuloma, resulting in potentially grave consequences. Bone wax may also lower bacterial clearance, thereby increasing risk of infection when attached to bone surfaces. Further, bone wax cannot be used in minimally invasive surgery procedures and firm pressure is required to secure hemostasis by a tamponade effect of the wax. The tamponade effect may be difficult to achieve in deep seated bleeding.

With regard to the third and fourth groups mentioned above, although the direct cost of purchasing the bone hemostatic agent is only fractional when compared to the cost of the surgical procedure, secondary cost due to complications arising from improper use of bone wax or its water soluble substitutes could amount to significant economic implications in a wide field of surgical practice including orthopaedics, maxillofacial and reconstructive/plastic surgery. Failure of joint fusion or pseudoarthrosis, thromboembolism, and necrotizing granuloma formation could lead to repeated surgeries and prolonged hospitalization.

In addition to the hemostatic efforts described above, it may also be desirable to enhance bone growth/regeneration.

However, the recent increase in the use of growth factors like bone morphogenetic proteins (BMPs) as an alternative to traditional bone graft material presents a new challenge. Blood oozing from bony surfaces could result in the “washing away” of growth factors delivered on collagen sponges that are commonly assembled with structural scaffold devices to enhance bone healing. This situation may cause failure of bone healing or pseudarthrosis as well as excessive bone formation at adjacent or ectopic sites. To combat this challenge, excessively large doses of growth factors are commonly advocated in order to compensate for such wastage. The excessively large doses create complications such as delayed osteolysis, excessive tissue swelling and hematoma which could necessitate further surgeries, leading to secondary costs.

Furthermore, minimally invasive surgery is preferred in many orthopaedic practices, such as chronic or debilitating low back pain and spinal deformities, due to its cosmetic advantage and other advantages. Hence, bone grafts typically applied over a broad surface area in open surgery are not possible in minimally invasive surgery.

There is therefore a need to provide a material that overcomes, or at least ameliorates, one or more of the disadvantages described above.

There is a need to provide a material and a method that can arrest bleeding from bony surfaces and simultaneously enhance bone regeneration.

### SUMMARY

In a first aspect, there is provided a bone filler material comprising a plurality of particles, each particle comprising a biodegradable outer shell and an inner core.

Due to the biodegradable outer shell, the disclosed material is advantageously biocompatible and the risk of rejection when implanted in a mammalian body can thereby be lowered. Further, due to the biodegradable outer shell, the disclosed material advantageously does not obstruct new bone formation.

In an embodiment, the biodegradable outer shell is a bone regeneration material. The disclosed material is therefore capable of plugging blood flow and simultaneously promoting bone regeneration when implanted in a mammalian body.

In some disclosed embodiments, the inner core comprises one or more bioactive molecules. Advantageously, the disclosed material is capable of delivering bioactive molecules into the body.

In a second aspect, there is provided an implant obtained by providing the material as disclosed herein at an injury site in a mammal.

In a third aspect, there is provided the use of the material as disclosed herein for preparing an implant for a mammal.

In a fourth aspect, there is provided a method of treating bone injury, the method comprising administrating the material as disclosed herein to an injury site.

In a fifth aspect, there is provided a method of stopping bone bleeding, the method comprising administrating the material as disclosed herein to the bleeding site.

In some disclosed embodiments, the disclosed material may be in powder form, or may be moldable into different shapes, or may be injectable, or may be provided as combinations thereof. Accordingly, the disclosed material may be easy to use and can be easily implanted or administered directly at the defective site. The disclosed material may thus be capable of use in minimally invasive surgery.

In a sixth aspect, there is provided the material as disclosed herein for treating bone injury.

In a seventh aspect, there is provided the material as disclosed herein for stopping bone bleeding.

In an eighth aspect, there is provided a method of preparing a bone filler material comprising the step of: aggregating a plurality of particles, each particle comprising a biodegradable outer shell and an inner core, to thereby form said bone filler material.

### DEFINITIONS

The following words and terms used herein shall have the meaning indicated:

As used herein, the term “bone” refers to rigid organs that constitute part of the endoskeleton of vertebrates and is taken to encompass hard tissue. Exemplary hard tissues include bone, calcified cartilage, and tissue that has become mineralized. Exemplary hard tissues also include long bone, maxillary bone, mandibular bone, and membranous bone. Exemplary hard tissues also include tibia, femur, shoulder, small joints, skull, and metatarsal. Exemplary hard tissues also include spine.

As used herein, the term “biodegradable” refers to materials that are bioresorbable and/or degrade and/or break down by mechanical degradation upon interaction with a physiological environment into components that are metabolizable or excretable, over a period of a few hours, for example less than 24 hours, or a few months, for example less than 6 months.

As used herein, the term “bioactive” refers to any biologically active molecule.

As used herein, the term “controlled release”, or grammatical variants thereof, refers to the release of a material (e.g. bone morphogenetic proteins) at such a rate that blood (e.g. plasma) concentrations are maintained within the therapeutic range but below toxic concentrations over a period of a few hours, for example 2 hours, or 3 hours, or a few days, for example 30 days or 60 days. The term “controlled release” encompasses all kinds of controlled release, including slow release, sustained and delayed release.

As used herein, the term “regeneration” in relation to tissue refers to the promotion of a process in which cells around an injured tissue proliferate and migrate into the lesion site to restore lost or dead tissues, when such tissues are partially lost or die due to a disease or injury.

As used herein, a particle size may refer to the diameter of the particles where they are substantially spherical. The particles may be non-spherical and the particle size range may refer to the equivalent diameter of the particles relative to spherical particles or may refer to a dimension (length, breadth, height or thickness) of the non-spherical particle.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, un-recited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DISCLOSURE OF OPTIONAL EMBODIMENTS

Exemplary, non-limiting embodiments of a bone filler material will now be disclosed.

In an embodiment, there is provided a bone filler material comprising a plurality of particles, each particle comprising a biodegradable outer shell and an inner core.

When implanted into a defect in a body, the disclosed bone filler material is capable of filling up the cavity of the defect. As the defective tissue may also bleed from a cavity of the defect, the disclosed material is capable of plugging blood flow by filling up the cavity.

The disclosed material also acts as a scaffold to promote and support bone regeneration. When implanted into a defect in a body, the outer shell is degradable, thereby allowing the defective tissue to grow on the disclosed material and regenerate itself.

When implanted, the scaffold provides structural support to allow the neighboring cells to adhere, proliferate and grow thereon. As the outer shell degrades upon interaction with the physiological environment, the bioactive molecules, when present in the inner core, may be controllably released and diffuse into the surrounding and new cells. Accordingly, the scaffold acts as a depot for bioactive molecules for subsequent controlled release delivery.

The disclosed material may therefore be useful in restoring or repairing a defect at a bony site. The disclosed material may enhance or promote hard tissue regeneration, such as spinal fusion and other regeneration of bone defects, with or without loading of bioactive molecules in the inner core. The disclosed material may exhibit increased osteogenic properties, increased hemostasis as well as reduced risk of infection at the surgical site.

Each particle comprises a biodegradable outer shell and an inner core and is therefore referred to herein as “a yolk/shell particle”. In embodiments, the yolk/shell particle is composed of a hollow outer shell that encapsulates an inner core.

Without being bound by theory, upon implantation, the interstitial space between the hollow outer shell and the inner core forms a porous capsule wall. Upon implantation, the outer shells of the plurality of particles fuse together to form the capsule wall encapsulating a plurality of inner cores. The implanted capsule wall is therefore formed from the biodegradable outer shells to provide structural support for the cells. The capsule wall is therefore degradable to thereby expose the inner cores to the implanted environment. Where the inner core comprises bioactive molecules, the release of the encapsulated bioactive molecules, such as thrombin, is facilitated by the exposed inner cores and thus, the release may advantageously be easy and controlled. Growth factors may also be encapsulated in the core to be released at a suitable time to improve the bone formation.

The outer shell may be bioactive, bio-transformable, biodegradable or combinations thereof. “Bio-transformable” means that the outer shell can react with chemical compounds to form a component in natural tissue. For example, the outer shell may comprise osteoconductive agents, such as apatite crystals, that mimic the composition of bone. Therefore, the outer shell may be capable of forming a component of bone and thereby regenerate bone cells.

The outer shell and inner core may be selected according to the properties desired. The materials chosen for the outer shell and inner core may be biomimetic in nature so as to suppress immune rejection.

The biodegradable outer shell may be comprised of a suitable material to achieve desired functions of each particle. The biodegradable outer shell may be comprised of a scaffold. The biodegradable outer shell or scaffold may be comprised of a bone regeneration material. The biodegradable outer shell or scaffold may be a bone regeneration material. The bone regeneration material is capable of inducing or promoting formation of bone. The bone regeneration material may be selected from the group consisting of: a calcium-containing compound, a bone morphogenic protein, hydroxyapatite, a derivative thereof and a mixture thereof.

In an embodiment, the biodegradable outer shell comprises a calcium-containing compound. The calcium-containing compound may aid in osteogenesis. The calcium-containing compound may be selected from the group consisting of: calcium carbonate, calcium phosphate, calcium sulphate, dicalcium phosphate dihydrate, dicalcium phosphate, tricalcium phosphate, tetracalcium phosphate, calcium bicarbonate, calcium acetate, calcium citrate, calcium silicate, calcium silicate hydrate, dicalcium citrate, dicalcium phosphate, monocalcium citrate, monocalcium phosphate, octacalcium phosphate, calcium peroxide, calcium pyrophosphate, calcium oxide, calcium oxalate, calcium nitrite, calcium nitrate, calcium lactate, calcium lactate gluconate, calcium magnesium acetate, calcium hydroxide, calcium gluconate, calcium glucoheptonate, calcium ascorbate and other calcium based materials beneficial to bone regeneration.

The biodegradable outer shell or scaffold may be dense or porous. The porosity or denseness of the biodegradable outer shell is a consequence of the preparation methods of the outer shell and the nature of the materials of the outer shell being used. Exemplary preparation methods of the outer shell and the disclosed material are disclosed herein. In embodiments, the biodegradable outer shell or scaffold is dense. The dense shell may be impenetrable to biological tissue. The dense shell may shield the inner core from in-growth of unwanted tissue. Pore diameters of the dense shell may be smaller than 0.02 μm. Biological tissue may be penetrable upon degradation of the outer shell.

The inner core, or “yolk”, may be comprised of a solid phase material. The inner core may be comprised of a plurality of particles. The inner core may not fill the internal volume created by the hollow outer shell. Accordingly, there may be an interstitial space between the inner core and the internal wall of the outer shell. Consequently, the inner core may freely move within the hollow shell because of the void disposed between the inner core and the outer shell.

The disclosed material may be capable of functioning as an injectable and moldable scaffold, as well as a delivery vessel for bioactive molecules, such as osteoinductive and osteoconductive factors. The inner core of the disclosed material may be loaded with one or more bioactive molecules to provide additional therapeutic effects. Hence, each particle can be utilized as a reservoir containing a single or multiple bioactive molecules. When implanted into the body, the particle is capable of delivering the therapeutic agents to the target area.

As a consequence of the injectability of the material, the scaffold can be provided directly onto the desired site. Thus, the scaffold does not require complex features to enhance cell affinity or controlled release. Furthermore, the injectability of the material results in the applicability of minimally invasive procedures and reduces the need for surgical interventions.

The inner core may be comprised of a material suitable to controllably release the loaded bioactive molecules when implanted into defective tissue.

The inner core may be comprised of materials that can carry bioactive molecules. The inner core may be comprised of one or more polymers. The one or more polymers may be biocompatible. The inner core may be comprised of natural or synthetic polymers. A natural polymer is one that occurs naturally in animals. The polymer may be selected from the group consisting of: silk fibroin, collagen, gelatin, elastin, albumin, fibrin, wheat gluten, gliadins, soy protein, polysaccharides, bacterial polymers, blends of biodegradable polymers, aliphatic polyesters, aromatic copolyesters, polyamides, poly(ester-amide)s, polyurethanes, polyanhydrides, vinyl polymers, other blends of polymers and mixtures thereof. In an embodiment, the inner core comprises natural materials such as silk fibroin, and thereby provides good biocompatibility and reduced infections.

The inner core may comprise one or more bioactive molecules. Advantageously, an array of bioactive molecules with different properties can be simultaneously delivered into the body.

In this embodiment, the inner core may be made of a material suitable for carrying the bioactive molecules. As the bioactive molecules are encapsulated within the outer shell in these embodiments, the bioactive molecules within the inner core are protected by the outer shell, thereby overcoming the problem of the washing away of these molecules or the dilution of these molecules by blood. Advantageously, excessive amounts of bioactive molecules are not required, resulting in cost savings. Further advantageously, the bioactive molecules may be provided in appropriate pharmaceutical doses, in alignment with minimally invasive surgery procedures.

The bioactive molecule may be selected from the group consisting of: growth factors, hemostatic agents, osteoconductive agents, antibiotics, anti-cancer agents, drugs with specific functions, agents with specific functions and mixtures thereof.

The hemostatic agent may be selected from the group consisting of: thrombin, alkylene oxide copolymers, collagen, gelatin, cellulose, chitosan, fibrin sealant, epinephrine, pro QR powder, kaolinite, poly-N-acetyl glucosamine, microfibrillar collagen, mineral zeolite, silver nitrate, aluminum chloride, ferric subsulfate, acrylates, tranexamic acid, aminocaproic acid, desmopressin, hypotensive anesthesia, vitamin K, protamine and other hemostatic agents.

The growth factor may be selected from the group consisting of: bone morphogenetic protein, transforming growth factor, activin, parathyroid hormone, insulin-like growth factor, Adrenomedullin (AM), Angiopoietin (Ang), Autocrine motility factor, Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumor necrosis factor-alpha(TNF-α), Vascular endothelial growth factor (VEGF), Wnt Signaling Pathway, placental growth factor (PlGF), Foetal Bovine Somatotrophin (FBS), IL-1 (Cofactor for IL-3 and IL-6) for activating T cells, IL-2 (T-cell growth factor) for stimulating IL-1 synthesis or for activating B-cells and NK cells, IL-3 for stimulating production of all non-lymphoid cells, IL-4 (Growth factor for activated B cells, resting T cells, and mast cells), IL-5 for inducing differentiation of activated B cells and eosinophils, IL-6 (Growth factor for plasma cells) for stimulating Ig synthesis, IL-7 (Growth factor for pre-B cells), NELL-1, recombinant growth factors and other growth factors.

The osteoconductive agent may be a compound that facilitates the formation of bone structure. The osteoconductive agent may be selected from apatite or hydroxyapatite.

In an embodiment, the inner core comprises the hemostatic agent, thrombin, and the growth factor, recombinant human bone morphogenetic protein 2, thereby providing a hemostatic effect and augmenting bone formation.

An appropriate dose of bioactive molecule to be loaded is dependent on the type of bioactive molecule used and will be apparent to a person of skill in the art. An exemplary dose includes about 1 mg/mL, or about 1.5 mg/mL, or about 2 mg/mL of BMP-2 for use in therapy of bone defects, or for use in therapy of bone injury, or for use in therapy of bone bleeding. Another exemplary dose includes about 4 rag, or about 5 μg, or about 6 μg of BMP-2 for use in therapy of bone defects, or for use in therapy of bone injury, or for use in therapy of bone bleeding, wherein the bone has a theoretical cylindrical volume of about 60 mm³.

In embodiments, the disclosed material, when implanted, is capable of cellular interaction, cellular bio-distribution and transduction of cell signaling by releasing the bioactive molecules comprised in the inner core, such as osteogenic growth factors and thrombogenic agents.

The thickness of the biodegradable outer shell may be controlled according to the degradation rate required. The biodegradable outer shell may have a thickness ranging from about 0.1 μm to about 5 μm, or about 0.1 μm to about 4 μm, or about 0.1 μm to about 3 μm, or about 0.2 μm to about 5 μm, or about 0.3 μm to about 5 μm, or about 0.4 μm to about 5 μm, or about 0.5 μm to about 5 μm, or about 0.5 μm to about 5 μm, or about 0.5 μm to about 4 μm, or about 0.5 μm to about 3 μm, or about 1 μm to about 5 μm, or about 2 μm to about 4 μm, or about 2 μm to about 5 μm, or about 1 μm to about 3 μm.

If the thickness of the outer shell is too thin, e.g. below about 0.1 μm, the shell structure will collapse. If the thickness of the outer shell is too thick, e.g. above about 5 μm, the degradation time of the outer shell will increase, resulting in an increase in time for the defective tissue to grow thereon and regenerate itself. An increase in degradation time may also result in an unfavorable increase in time to release the bioactive molecules when comprised in the inner core. In an embodiment, the biodegradable outer shell may have a thickness ranging from about 0.5 μm to about 3 μm, or from about 1 μm to about 3 μm.

The inner core may have a diameter ranging from the nano-size range to the micro-size range. The inner core may have a diameter ranging from about 100 nm to about 45 μm, about 100 nm to about 30 μm, or about 200 nm to about 45 μm, or about 200 nm to about 30 μm, or about 300 nm to about 45 μm, or about 300 nm to about 30 μm, or about 500 nm to about 45 μm, or about 500 nm to about 30 μm, or about 1 μm to about 30 μm, or about 5 μm to about 30 μm, or about 10 μm to about 30 μm, or about 10 μm to about 45 μm.

Where the inner core is comprised of a plurality of particles, each of such particle may have a size of from about 0.2 μm to about 3 μm.

The ratio of the diameter of the inner core to the thickness of the outer shell may be selected to be in the range of from about 450:1 to about 1:50, or about 100:1 to about 1:10, or 1:1, or 9:1.

The ratio of the diameter of the inner core to the diameter of the particle comprising the biodegradable outer shell and the inner core may be selected to be in the range of from about 0.7 to about 0.9.

The disclosed material may be packaged in powder form. The size of the powder particles may be suitable for uptake by the defective tissue. The powder particles may be an aggregate of a plurality of the particles comprising a biodegradable outer shell and an inner core. The powder particles may be a powdered aggregate of a plurality of the particles comprising a biodegradable outer shell and an inner core. The powder particles may have a diameter ranging from about 12 μm to about 50 μm.

The disclosed material may be packaged in a bulk form, such as a putty or paste or gel. The material may be moldable into different geometrical shapes. The material may be moldable into any convenient shape and size suitable for implanting into the defective tissue to fill irregular defects. The disclosed scaffold material is thus flexible and suitable for filling up irregular bone defects. The shapes and sizes of the material are not limited, so long as the material can fulfil its purpose of hemostasis and osteogenesis.

The material may be shaped into a disk having a diameter of any size, depending on the defect and/or the defective site.

The material may be shaped into a cylinder of any length, depending on the defect and/or the defective site.

The disclosed material may be injectable. As the material may be in the form of putty which is injectable and/or moldable, the disclosed material may be provided in a syringe or any other suitable injectable container that can eject the material into the desired shape. The implant in the body will therefore be of the desired shape upon injection.

The disclosed material may be provided to a defective site in an amount suitable to for its purpose, e.g. to fill up a cavity, to promote bone cell growth, etc. An appropriate amount of material to be provided will be apparent to a person of skill in the art.

In embodiments, the disclosed bone filler material may be used for preparing an implant for a mammal. The implant may be obtained by providing the disclosed material at an injury site or a bleeding site in a mammal.

The material may be provided directly onto a defective hard tissue. In an example, the material may be implanted into a cavity of a defective hard tissue using a syringe.

In embodiments, there are provided methods of treating bone injury or stopping bone bleeding, the methods comprising administrating the material disclosed herein to an injury site or the bleeding site, as the case may be.

Upon implantation, the material may be contacted with a chemical compound to allow the outer shells of each particle to react and fuse together. The fusion of the particles results in a scaffold for the cells to grow thereon and regenerate themselves.

The material may be incubated in a solution of the chemical compound capable of causing the fusion of the outer shells of each particle comprising the outer shell and inner core, to form the capsule wall of the implant. In an embodiment, the chemical compound is a phosphate-containing solution. In this embodiment, the phosphate reacts with the calcium of the outer shell to produce calcium phosphate. During the transformation to calcium phosphate, the voids or interstitial spaces disposed between the outer shells and the inner cores of each particle are preserved. The capsule wall of the implant therefore encapsulates the inner cores, wherein the inner cores may freely move within the capsule wall due to the preserved voids.

The capsule wall of the implant may have pores. The pore sizes are not limited and may be in the range of tens of μm to hundreds of μm.

Degradation of the outer shell or capsule wall allows body fluid to penetrate the void and reach the inner core. In the embodiments where the inner core comprises bioactive molecules, the bioactive molecule comprised in the inner core may be controllably released to the injury site or bleeding site by diffusion. The bioactive molecules diffuse out of the material of the inner core and into the body fluid to be delivered to the desired site. Body fluid may also penetrate the inner core to be mixed with the bioactive molecules that may be loaded therein. Accordingly, without being bound by theory, the bioactive molecules may be bioactive within the inner core, before being released from the inner core into the site of implant.

The speed of the release of bioactive molecules is dependent on the speed of degradation of the inner core. A person skilled in the art would understand which core materials are suitable for desired release times. For example, when the inner core is comprised of silk fibroin having BMP-2 loaded therein, BMP-2 can be controllably released for more than about 30 days.

The phosphate-containing solution may be presented at the injury or bleeding site.

The defects may be filled as the porous scaffold is formed. The filler material subsequently aids in hemostasis by a tamponade effect. Further, as cells are able to grow on the scaffold, tissue regeneration may be enhanced even without loading of bioactive molecules such as growth factors.

In embodiments, there is provided a method of preparing a bone filler material comprising the step of: aggregating a plurality of particles, each particle comprising a biodegradable outer shell and an inner core, to thereby form said bone filler material.

Prior to the aggregating step, the method may comprise a step of loading the inner core into the biodegradable outer shell.

In an embodiment, the step of loading comprises mixing the inner core with a mixture of a precursor of the biodegradable outer shell, a solvent and a micelle-forming compound.

The micelle-forming compound may be a surfactant or an amphiphilic polymer.

The surfactant may be selected from cationic, anionic, amphoteric, zwitterionic or non-ionic surfactants, or combinations thereof. The surfactant may be selected from the group consisting of the surfactant is selected from the group consisting of anionic surfactants such as cetyl trimethylammonium bromide, alkyl sulfates, alkyl ether sulfates, alkyl ester sulfonates, alpha olefin sulfonates, linear alkyl benzene sulfonates, branched alkyl benzene sulfonates, linear dodecylbenzene sulfonates, branched dodecylbenzene sulfonates, alkyl benzene sulfonic acids, dodecylbenzene sulfonic acid, sulfosuccinates, sulfated alcohols, ethoxylated sulfated alcohols, alcohol-sulfonates, ethoxylated and propoxylated alcohol sulfonates, alcohol ether sulfates, ethoxylated alcohol ether sulfates, propoxylated alcohol sulfonates, sulfated nonyl phenols, ethoxylated and propoxylated sulfated nonyl phenols, sulfated octyl phenols, ethoxylated and propoxylated sulfated octyl phenols, sulfated dodecyl phenols, ethoxylated and propoxylated sulfated dodecyl phenols and alpha olefin sulfates; nonionic surfactants such as amine oxides, ethoxylated or propoxylated nonyl phenols, ethoxylated or propoxylated alkyl phenols, ethoxylated or propoxylated octyl phenols, ethoxylated or propoxylated dodecyl phenols, ethoxylated or propoxylated primary linear alcohols from C₄ to C₂₀₊, polyethylene glycols of all molecular weights and reactions and polypropylene glycols of all molecular weights and reactions; and hydrotropic surfactants such as dicarboxylic acids, phosphate esters, sodium xylene sulfonate, and sodium dodecyl diphenyl ether disulfonate.

The amphiphilic polymer may be selected from the group consisting of polyvinyl alcohol, polyethylene glycol, polysaccharide, polyacrylic acid, a copolymer of polyacrylic acid, polymethacrylic acid, a copolymer of polymethacrylic acid, alginate, chitosan, starch and gelatin.

The solvent may be selected from an aqueous solvent or an organic solvent, depending on the solubility of the micelle-forming compound.

The organic solvent may be selected from the group consisting of methane, ethane, propane, butane, pentane, hexane, heptane, octane, nonane, decane and other organic solvents not miscible in water. The aqueous solvent may be water.

The micelle-forming compound may be mixed at a concentration of about 0.01 g/ml to about 1.5 g/ml, 0.02 g/ml to about 1.5 g/ml, 0.03 g/ml to about 1.5 g/ml, 0.04 g/ml to about 1.5 g/ml, 0.05 g/ml to about 1.5 g/ml, or about 0.075 g/ml to about 1.5 g/ml, or about 0.1 g/ml to about 1.5 g/ml, or about 0.15 g/ml to about 1.5 g/ml, or about 0.2 g/ml to about 1.5 g/ml, or about 0.3 g/ml to about 1.5 g/ml, or about 0.01 g/ml to about 1.25 g/ml, or about 0.01 g/ml to about 1 g/ml, or about 0.05 g/ml to about 1.25 g/ml, or about 0.05 g/ml to about 1 g/ml, or about 0.015 g/ml to about 1 g/ml, or about 0.05 g/ml to about 0.75 g/ml, or about 0.05 g/ml to about 0.5 g/ml.

Upon mixing with the micelle-forming compound, the micelles formed may stabilize the precursor of the outer shell and the inner core. The micelles formed may be in the form of a vesicle. The vesicles may encapsulate the inner core. The solvent used may aid in arranging the inner core on the inside of the formed micelles and the precursor on the outside of the formed micelles. For example, carbonate anions from the calcium carbonate outer shell are attracted to the periphery of the cetyl trimethylammonium bromide vesicle and will form a shell around the vesicle encapsulating the inner core.

Micelles or vesicles may be formed when the concentration of the surfactant is above its critical micelle concentration. In embodiments, the critical micelle concentration ranges from about 10⁻³ M to about 10⁻² M.

The mixing step may be conducted at a temperature suitable to form micelles. When the solvent is an aqueous solvent, the temperature of the mixing step may be conducted at about 30° C. to about 50° C.

The precursor of the biodegradable outer shell may be calcium chloride.

The method may comprise adding a chemical compound to convert the precursor of the outer shell into the biodegradable outer shell. The chemical compound may be selected according to the precursor used. The chemical compound may be a carbonate-containing compound, soluble in water. In the embodiment where the calcium precursor is calcium chloride, the chemical compound may be sodium carbonate.

The compound to convert the precursor may be added to the mixture in a concentration of from about 1M to about 10M. The calcium precursor may be added to the mixture in a concentration of from about 5M to about 15M.

The compound to convert the precursor may be added dropwise to allow time to grow the crystals of the outer shell. The time taken to add the compound to convert the precursor may be at least 6 hours, or at least 10 hours, or at least 12 hours.

The micelles may be washed away, thereby leaving a void between the inner core and outer shell.

The method may comprise the step of loading one or more bioactive molecules into the inner core. The one or more bioactive molecules may be loaded in the inner core by absorption and electrostatic interaction.

The step of loading one or more bioactive molecules into the inner core may comprise mixing said one or more bioactive molecules with the inner core prior to the step of loading the inner core into the biodegradable outer shell.

The method may also comprise the step of selecting a bone regeneration material for said biodegradable outer shell.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a schematic diagram of a method of preparing a bone filler material according to an embodiment of the present disclosure.

FIG. 2 is a micrograph of a particle according to an embodiment of the present disclosure.

FIG. 3a is a photograph of the bone filler material, according to an embodiment of the present disclosure, in various shapes.

FIG. 3b is a photograph of a surgical process before implantation of the bone filler material, according to an embodiment of the present disclosure, into bone defects.

FIG. 3c is a photograph of a surgical process after implantation of the bone filler material, according to an embodiment of the present disclosure, into bone defects.

FIG. 4a is an X-ray μ-computed tomography (μ-CT) image of the iliac bone of rabbits after application of the putty of Example 3 on defects of the iliac bone.

FIG. 4b is an X-ray μ-computed tomography (μ-CT) image of the iliac bone of rabbits after application of bone wax referred to in Example 3 on defects of the iliac bone.

FIG. 4c is an X-ray μ-computed tomography (μ-CT) image of the iliac bone of rabbits after application of collagen/BMP-2 referred to in Example 3 on defects of the iliac bone.

### DETAILED DESCRIPTION OF DRAWINGS

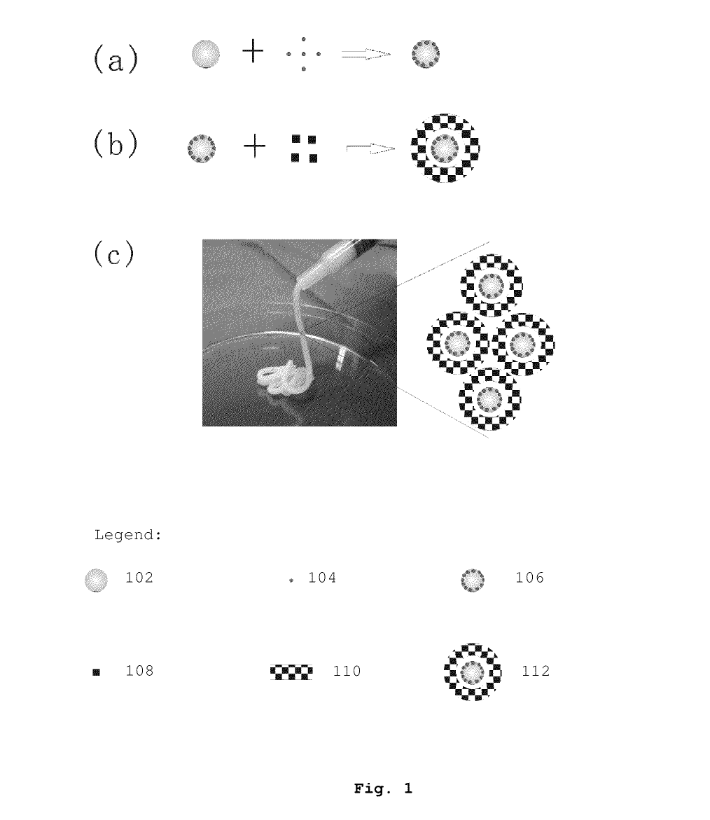

A schematic diagram of an embodiment of the disclosed method of preparing a bone filler material loaded with bioactive molecules is shown in FIG. 1. The bone filler material comprises a plurality of particles, wherein each particle 112 is of a yolk/shell configuration. The yolk 106 (or inner core) is a polymeric particle 102 comprising bioactive molecules 104. The shell 110 is a calcium-containing compound that encapsulates the yolk 106.

In step (a), the polymeric particle 102 is functionalized by bioactive molecules 104 to form a bioactive functionalized polymeric particle, i.e. yolk 106. The bioactive molecules 104 are capable of controllably releasing biological precursors.

In step (b), the yolk 106 is mixed with calcium precursors 108 and other additives in the presence of a solvent. The calcium precursors 108 are aided by the additives to encapsulate the yolk 106 and form particle 112, which is an annular calcium shell 110 encrusting the yolk 106. As the calcium shell 110 annularly encapsulates the yolk 106, there is a space between the yolk 106 and the annular calcium shell 110 such that the yolk 106 is movable within the shell 110.

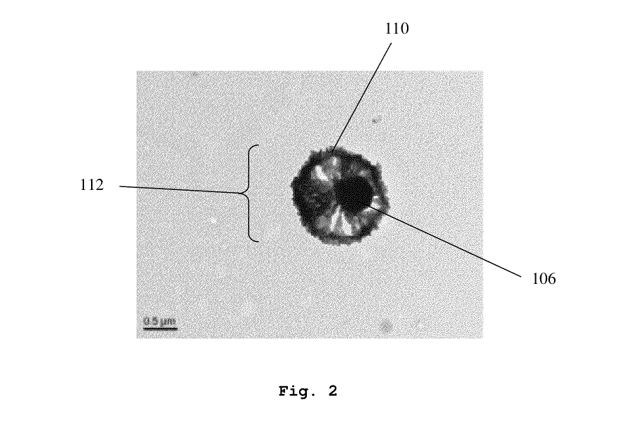

A micrograph of the particle 112 is shown in FIG. 2. It can be seen that particle 112 comprises an outer shell 110 and an inner core 106. Particle 112 has a diameter of about 1.5 μm, and the inner core 106 has a diameter of about 0.5 μm. The outer shell 110 has a thickness of about 0.1 μm.

Referring again to FIG. 1, in step (c), a plurality of particles 112 is aggregated into an injectable and moldable putty. The aggregated particles are capable of forming a scaffold to support bone growth when implanted into the body.

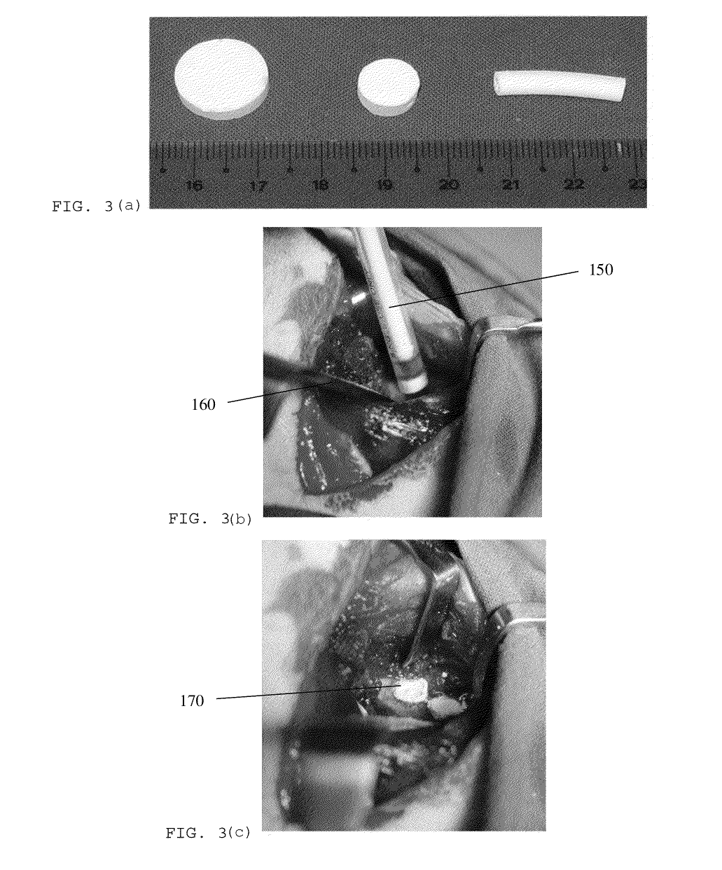

The disclosed bone filler material can be molded into various shapes and sizes as appropriate for implantation into bone defects. As shown in FIG. 3(a), the disclosed material can be shaped into a disk or a cylinder. The disks shown in FIG. 3(a) have a diameter of about 1.5 cm and 1 cm, while the cylinder shown in FIG. 3(a) has a length of about 2 cm.

In use, the disclosed bone filler material can be injected directly to the site in need, e.g. a bleeding site or an injured site. As seen in FIG. 3(b), an incision is made by scalpel 160 to expose and identify the part in need of healing. A syringe 150 is loaded with the disclosed material and placed above the defective bone. It can be appreciated that any container matching the volume and/or shape of the cavity in the defective bone, other than a syringe, can be used. The material is injected directly into the bone defect in a minimally invasive surgical process. The implanted material 170 is able to fill up the cavity and effectively stop the bleeding, as shown in FIG. 3(c).

### EXAMPLES

Non-limiting examples of the invention and comparative examples will be further described in greater detail by reference to specific Examples, which should not be construed as in any way limiting the scope of the invention.

### Example 1

In this example, surfactant templates in both organic and aqueous systems were used to prepare the bone filler material. Silk fibroin nanoparticles were used as the inner core.

Organic System

Sodium dodecyl sulphate (SDS, 10 g) was used as the surfactant template and was dissolved in n-heptane (100 ml). After the SDS was fully dissolved, sodium carbonate (1M, 10 ml) was slowly added into the organic n-heptane solution. The emulsion was mixed with a mechanical overhead stirrer.

Thereafter, 100 μL 2% silk fibroin nanoparticles followed by calcium chloride (10M, 1 ml) as the calcium precursor were added into the heptane solution.

The emulsion was mixed for at least 1 hour and centrifuged to produce the yolk/shell particles as a putty.

Aqueous System

SDS (0.5 g) and cetyl trimethylammonium bromide (CTAB, 1 g) were used as the surfactant template.

SDS and CTAB were dissolved in 100 ml of distilled water. The solution was heated to 45° C. for 30 mins to ensure that micelles were forming in the solution.

Thereafter, 100 μL 2% silk fibroin nanoparticles followed by calcium chloride (10M, 1 ml) as the calcium precursor were added into the aqueous solution. The surfactant micelles stabilized the aqueous calcium chloride on the outside and the silk fibroin nanoparticles on the inside.

Sodium carbonate (1M, 10 ml) was added into the solution drop by drop to provide enough time for the calcium carbonate crystals produced to grow on the micelle surfactant template.

The emulsion was mixed for at least 1 hour and centrifuged to produce the yolk/shell particles as a putty.

### Example 2

In this example, polymer templates in organic systems were used to prepare the bone filler material.

Polyvinyl alcohol (PVA) was used as the amphiphilic polymer template to form polymer micelles in an organic phase. Silk fibroin nanoparticles were used as the inner core.

PVA (2 g) was mixed with 100 μL 2% silk fibroin nanoparticles and calcium chloride solution (10M, 10 ml). The solution was added into 100 ml chloroform.

The resultant emulsion was agitated. The amphiphilic polymer micelles stabilized the aqueous calcium chloride on the outside and the silk fibroin nanoparticles on the inside.

Sodium carbonate (10M, 1 ml) was added into the emulsion drop by drop for at least 12 hours to provide enough time for the calcium carbonate crystals produced to grow on the micelle polymer template.

The emulsion was mixed for at least 12 hours and centrifuged to produce the yolk/shell particles as a putty.

### Example 3

Blank putty was prepared and applied to defects in the iliac bone of rabbits. As a comparison, bone wax and collagen/BMP-2 (INFUSE® Bone Graft, Medtronic, Memphis, USA) were similarly applied to defective iliac bone of rabbits.

The blank putty comprises yolk/shell particles. Dry yolk/shell particle powder was mixed with distilled water (95 to 5, W/W) to form the putty. The putty was then squeezed into the defects.

100 mg of the blank putty were implanted into each defect. For the control groups, enough bone wax and collagen were implanted to fill in each defect.

5 μg of BMP-2 were applied in each defect wherever necessary.

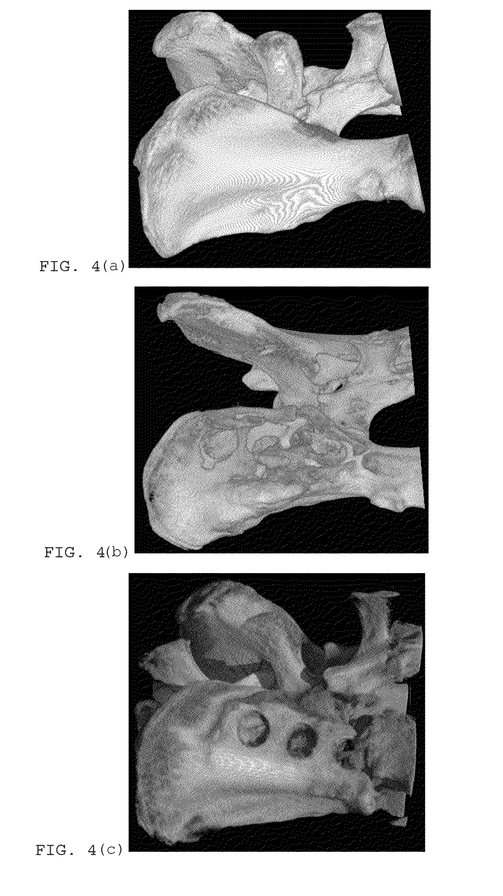

After 4 weeks from application, the results are shown in FIGS. 4a-c. As can be seen in FIG. 4a, the blank putty promoted bone healing and regeneration more significantly than the bone wax or collagen/BMP-2, even without loading of any growth factor in the putty. There was complete bone healing and recovery as no further bone defects could be observed.

FIG. 4b shows that bone defect regeneration was hampered by the bone wax and thus there was no recovery in the cavities of the defective bone. Instead, the bone wax caused bone deformation.

Most interestingly, the INFUSE® Bone Graft was used as a control, but only a small amount of bone regeneration could be observed (FIG. 4c).

Therefore, the product disclosed herein is evidenced to significantly enhance bone healing.

### APPLICATIONS

The disclosed material is capable of overcoming uncertainties in achieving bone regeneration, such as spinal fusion, in minimally invasive surgery (MIS) due to its biodegradable properties.

In addition to MIS, the disclosed material also finds application in open surgeries involving joint fusion as well as in delayed or failed bone union. Furthermore, since the disclosed material can also act as a growth factor carrier system, applications can be found in segmental bone defects, and in maxilla-facial and cranial reconstructive surgery. The disclosed material may therefore act as a bone hemostatic agent as well as a synthetic autograft bone substitute. The disclosed material can be applied in orthopedic reconstructive areas of the spinal fusion and other regeneration of bone defects.

The disclosed material has the potential to reduce the MIS procedures that are converted to open surgery due to uncontrollable bleeding in skeletal reconstruction. The disclosed material controls blood loss and can deliver a concentration of bone growth factors and/or bone forming cells at the same time to a localized area. Thus, a patient can benefit fully from the advantages of minimally invasive surgery without the need for additional bone grafts, thereby resulting in less pain, less blood loss, faster recovery, shorter surgery times and shorter hospitalization times.

The disclosed material acts as structural support to support bone regeneration when implanted. The implant may be capable of hemostasis and may promote the regeneration of bone cells. The implant may also be used as a bulking agent. The disclosed material may find use as a tissue sealant (with both chemical hemostatic and physical tamponade effects), a growth factor and hemostatic agent co-delivery vehicle, and bone void filler. The disclosed material may find use in orthopaedic reconstructive areas of the spinal fusion and other bone defects regeneration.

The surgeons can also benefit since the disclosed material is easy to use, similar to the existing use of bone wax. Without the need for additional bone grafts, time is saved.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

What is claimed is:

1. A bone filler material comprising a plurality of particles, each particle comprising a biodegradable outer shell and an inner core.

2. The material of claim 1, wherein said biodegradable outer shell is comprised of a scaffold.

3. The material of claim 2, wherein the scaffold is comprised of a bone regeneration material selected from the group consisting of: a calcium-containing compound, a bone morphogenic protein, hydroxyapatite, a derivative thereof and a mixture thereof.

4. The material of claim 3, wherein the calcium-containing compound is selected from the group consisting of: calcium carbonate, calcium phosphate, calcium sulphate, dicalcium phosphate dihydrate, dicalcium phosphate, tricalcium phosphate, tetracalcium phosphate, calcium bicarbonate, calcium acetate, calcium citrate, calcium silicate, calcium silicate hydrate, dicalcium citrate, dicalcium phosphate, monocalcium citrate, monocalcium phosphate, octacalcium phosphate, calcium peroxide, calcium pyrophosphate, calcium oxide, calcium oxalate, calcium nitrite, calcium nitrate, calcium lactate, calcium lactate gluconate, calcium magnesium acetate, calcium hydroxide, calcium gluconate, calcium glucoheptonate and calcium ascorbate.

5. The material of claim 2, wherein the scaffold is dense or porous.

6. The material of claim 1, wherein said inner core is comprised of a solid phase material.

7. The material of claim 1, wherein said inner core comprises one or more synthetic polymers or polymers that occur naturally in animals, wherein the one or more polymers are selected from the group consisting of: silk fibroin, collagen, gelatin, elastin, albumin, fibrin, wheat gluten, gliadins, soy protein, polysaccharides, bacterial polymers, blends of biodegradable polymers, aliphatic polyesters, aromatic copolyesters, polyamides, poly(ester-amide)s, polyurethanes, polyanhydrides, vinyl polymers and mixtures thereof.

8. The material of claim 1, wherein said inner core comprises one or more bioactive molecules.

9. The material of claim 8, wherein the bioactive molecule is selected from the group consisting of: growth factors, hemostatic agents, osteoconductive agents, antibiotics, anti-cancer agents and mixtures thereof.

10. The material of claim 8, wherein the hemostatic agent is selected from the group consisting of: thrombin, alkylene oxide copolymers, collagen, gelatin, cellulose, chitosan, fibrin sealant, epinephrine, pro QR powder, kaolinite, poly-N-acetyl glucosamine, microfibrillar collagen, mineral zeolite, silver nitrate, aluminum chloride, ferric subsulfate, acrylates, tranexamic acid, aminocaproic acid, desmopressin, hypotensive anesthesia, vitamin K and protamine.

11. The material of claim 8, wherein the growth factor is selected from the group consisting of: bone morphogenetic protein, transforming growth factor, activin, parathyroid hormone, insulin-like growth factor, Adrenomedullin (AM), Angiopoietin (Ang), Autocrine motility factor, Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumor necrosis factor-alpha(TNF-α), Vascular endothelial growth factor (VEGF), Wnt Signaling Pathway, placental growth factor (PlGF), Foetal Bovine Somatotrophin (FBS), IL-1 (Cofactor for IL-3 and IL-6) for activating T cells, IL-2 (T-cell growth factor) for stimulating IL-1 synthesis or for activating B-cells and NK cells, IL-3 for stimulating production of all non-lymphoid cells, IL-4 (Growth factor for activated B cells, resting T cells, and mast cells), IL-5 for inducing differentiation of activated B cells and eosinophils, IL-6 (Growth factor for plasma cells) for stimulating Ig synthesis, IL-7 (Growth factor for pre-B cells) and NELL-1.

12. The material of claim 8, wherein the osteoconductive agent is apatite or hydroxyapatite.

13. The material of claim 1, wherein the material is in powder form.

14. The material of claim 1, wherein the material is moldable into different shapes or injectable.

15. An implant obtained by providing the material of claim 1 at an injury site in a mammal.

16. The implant of claim 15, wherein the implant acts as a structural support to support bone regeneration and/or is capable of hemostasis and/or promotes the regeneration of bone cells.

17. A method of treating bone injury or stopping bone bleeding, the method comprising administrating the material of claim 1 to an injury or bleeding site.

18. The method of claim 17, comprising contacting the material of claim 1 with a phosphate-containing solution to form pores from a void disposed between the biodegradable outer shell and the inner core.

19. The method of claim 17, comprising controllably releasing the bioactive molecule comprised in the inner core to the injury site or bleeding site by diffusion.

20. The method of claim 18, wherein the bioactive molecule diffuses through the pores formed from the void disposed between the biodegradable outer shell and the inner core.
